(19) United States
(12) Patent Application Publication
(10) Pub. No.: US 2010/0047901 A1
(43) Pub. Date: Feb. 25, 2010

(54) SYSTEM AND METHOD FOR ELECTRONIC SENSING OF BIOMOLECULES

(75) Inventors: Keith Bradley, New York, NY (US); Jean-Christophe P. Gabriel, Le Charpen (FR); George Grüner, Los Angeles, CA (US); Alexander Star, Pittsburgh, PA (US); Eugene Tu, San Diego, CA (US); Christian Valcke, Orinda, CA (US)

Correspondence Address: Weaver Austin Villeneuve & Sampson LLP, P.O. BOX 70250, OAKLAND, CA, 94612-0250, US

(73) Assignee: Nanomix, Inc., Emeryville, CA (US)

(21) Appl. No.: 12/607,912

(22) Filed: Oct. 28, 2009

Related U.S. Application Data
(63) Continuation of application No. 11/259,414, filed on Oct. 25, 2005, now abandoned.
Application No. 11/259,414 is a continuation-in-part of application No. 10/704,066, filed on Nov. 7, 2003, now abandoned.
Application No. 10/704,066 is a continuation-in-part of application No. 10/345,783, filed on Jan. 16, 2003.
(60) Provisional application No. 60/622,468, filed on Oct. 25, 2004.
(60) Provisional application No. 60/660,441, filed on Mar. 10, 2005.
(60) Provisional application No. 60/668,879, filed on Apr. 5, 2005.
(60) Provisional application No. 60/669,126, filed on Apr. 6, 2005.
(60) Provisional application No. 60/424,892, filed on Nov. 8, 2002.
(60) Provisional application No. 60/349,670, filed on Jan. 16, 2002.

(51) Int. Cl. G01N 33/53

(52) U.S. Cl. 435/287.2

(57) ABSTRACT

A nanoelectronic device is combined with a cellular membrane component to provide a sensor for biomolecules or to provide information about the structure of the membrane. The nanoelectronic device may comprise a network of randomly-oriented nanotubes, or other nanostructure, arranged on a substrate with adjacent electrodes so as to operate as a field-effect transistor sensor or as a capacitive sensor. A cellular membrane is disposed over the nanostructure element.

### RELATED APPLICATIONS

This application claims priority pursuant to 35 U.S.C. §119(e) to U.S. Provisional Application Nos. 60/622,468 filed Oct. 25, 2004, 60/660,441 filed Mar. 10, 2005, 60/668,879 filed Apr. 5, 2005, and 60/669,126 filed Apr. 6, 2005, which applications are specifically incorporated herein, in their entirety, by reference.

This application is a continuation-in-part of co-pending U.S. application Ser. No. 10/704,066 filed Nov. 7, 2003, which claims priority to Provisional Application No. 60/424,892 filed Nov. 8, 2002, and which is a continuation-in-part of application Ser. No. 10/345,783 filed Jan. 16, 2003 (which claims priority to Provisional Application No. 60/349,670 filed Jan. 16, 2002), and application Ser. No. 10/656,898 filed Sep. 5, 2003 (which claims priority to Provisional Application No. 60/408,547 filed Sep. 5, 2002). Each of the foregoing provisional and non-provisional applications are specifically incorporated herein, in their entirety, by reference.

### GOVERNMENT RIGHTS

Portions of the work represented by the this application have been supported by grants sponsored by the United States, and the Government may have certain rights to invention disclosed herein.

### BACKGROUND OF THE INVENTION

1. Field of the Invention

The present invention relates to the detection of biological molecules by nanometer-scale electronic sensors.

2. Description of Related Art

Current biological sensing techniques commonly rely on optical detection principles that are inherently complex, require multiple steps between the actual engagement of the analyte and the generation of a signal, multiple reagents, preparative steps, signal amplification, complex data analysis and/or relatively large sample size.

Nanowires and nanotubes, by virtue of their small size, large surface area, and near one-dimensionality of electronic transport, are promising candidates for electronic detection of chemical and biological species. Field effect transistors (FETs) fabricated from component semiconducting single wall carbon nanotubes (NTs) have been studied extensively for their potential as sensors. A number of properties of these devices have been identified, and different mechanisms have been proposed to describe their sensing behavior. Devices that incorporate carbon nanotubes have been found to be sensitive to various gases, such as oxygen and ammonia, and these observations have confirmed the notion that such devices can operate as sensitive chemical sensors.

Single-walled nanotube (“SWNT”) devices, including field-effect transistors (“FET's”) and resistors, can be fabricated using nanotubes grown on silicon or other substrates by chemical vapor deposition from iron-containing catalyst nanoparticles with methane/hydrogen gas mixture at 900° C. Other catalyst materials and gas mixtures can be used to grow nanotubes on substrates, and other electrode materials and nanostructure configurations and have been described previously by Gabriel et al. in application Ser. Nos. 10/099,664 and 10/177,929, both of which are incorporated by reference herein. Currently, technology for constructing practical nanostructure devices is in its infancy. While nanotube structures show promise for use as sensor devices and transistors, current technology is limited in many ways.

For example, it is desirable to take advantage of the small size and sensitivity of nanotube and other nanostructure sensors to sense biological molecules, such as proteins. But a useful sensor of this type should selectively and reliably respond to a molecular target of a specific type. For example, it may be desirable to selectively sense a specific protein, while not responding to the presence of other proteins in the sample. Examples of covalent chemical attachment of biological molecules to nanotubes, including proteins and DNA, are known in the art, although it has not been convincingly demonstrated that useful detection of specific proteins or other large biomolecules can be accomplished in this way. For one thing, covalent chemical attachment has the disadvantage of impairing physical properties of carbon nanotubes, making structures of this type less useful as practical sensors. In addition, the carbon nanotubes are hydrophobic, and generally non-selective in reacting with biomolecules.

It is desirable, therefore, to provide a nanostructure sensing device, such as for example a nanotube device, that is biocompatible, and exhibits a high degree of selectivity to particular biomolecular targets.

### SUMMARY OF THE INVENTION

In accordance with embodiments of the present invention, a nanotube sensor architecture is provided, which allows the detection of protein-protein interactions and, at the same time, reduces or eliminates non-specific binding. The sensor may be operated as a nanostructure field effect transistor, to detect the presence of a specific protein or other biomolecule. Further provided are methods for making and operating the sensing device.

A nanostructure device according to the invention may comprise a nanotube, such as a carbon nanotube or network of nanotubes, disposed along a substrate, such as a silicon substrate. The nanotube structure may span two conductive elements, which may serve as electrical terminals, or as a source and drain. A passivation layer, such as of silicon monoxide, may be deposited over the conductive elements and a portion of the nanotube, leaving a portion of the nanotube between the conductive elements exposed. The nanotube may be coated with a thin polymer layer, for example comprising poly(ethylene imine) (“PEI”) and poly(ethylene glycol) (PEG). In this configuration, the device may be operated as an n-type FET, as further described in application Ser No. 10/656,898. Advantageously, the polymer layer is hydrophilic and biocompatible, making the nanotube device essentially non-reactive to large biomolecules such as proteins.

A bioreceptor layer may be attached over the polymer layer, configured for reactivity to a specific biomolecule. For example, biotin is known to selectively bind to streptavidin. The bioreceptor layer should be configured to bind to the polymer layer. For example, a solution of biotin-N-hydroxysuccinimide ester reacts with primary amines in PEI, thereby binding biotin molecules to the polymer layer. The bioreceptor layer may comprise a mono-molecular layer, comprised of discrete bioreceptor molecules attached to the polymer layer.

The resulting device will exhibit transconductance that varies depending on the presence of the targeted biomolecule in its sample environment. For example, a bioreceptor layer comprised of attached biotin molecules will selectively bind to streptavidin, causing a measurable decrease in transconductance at negative gate voltages. The device may therefore be used as a sensor for streptavidin. To sense other biomolecules, the device may be provided with a different bioreceptor layer that is configured to bind to the desired target.

In an embodiment of the invention, a cellular material may be operatively engaged with the nanoelectronic sensor. The cellular material may be selected for reactivity with a particular biomolecule or type of biomolecule. For example, a nanotube, network of nanotubes, or other nanostructure configured as a FET or other electronic device may be engaged with a portion of cellular membrane material. The cellular membrane may be applied over or on the nanostructure in different orientations, such as with the cytoplasmic side or the extracellular side oriented towards the nanostructure. The cellular membrane material exhibits selective reactivity towards biomolecules depending on its orientation and source, without disrupting the utility of the nanoelectronic device as a FET or capacitance sensor. Thus, the nanoelectronic sensor may be made to selectively respond to various biomolecules that are reactive with the cell membrane, or to biomolecules attached to the cell membrane, while not being triggered by non-targeted analytes.

A more complete understanding of the biomolecular sensor will be afforded to those skilled in the art, as well as a realization of additional advantages and objects thereof, by a consideration of the following detailed description of the preferred embodiment. Reference will be made to the appended sheets of drawings which will first be described briefly.

### BRIEF DESCRIPTION OF THE DRAWINGS

FIG. 1 is a is a schematic diagram of a nanotube field effect transistor (NTFET) configured as a biomolecule sensor according to the invention.

FIG. 2 is a flow chart showing exemplary steps of a method for making a nanotube biosensor according to the invention.

FIG. 3A is a schematic of a chemical scheme for bonding biotin to a PEI/PEG polymer layer over a nanotube.

FIG. 3B is a chart comparing transconductance of a native PEI/PEG-coated NTFET device with its transconductance after 1 hour, and after 18 hours of being reacted with biotin-N-hydroxysuccinimide ester.

FIG. 4 is a chart comparing transconductance of a bare NTFET to a NTFET coated with a PEI/PEG polymer layer and a NTFET with a biotinylated PEI/PEG layer.

FIG. 5 is a chart comparing the transconductance of a biotinylated, PEI/PEG-coated NTFET device, in the absence and presence of streptavidin.

FIG. 6 is a chart comparing the transconductance of a bare NTFET device, in the absence and presence of streptavidin.

FIG. 7 is a chart comparing the transconductance of a PEI/PEG-coated NTFET device without biotin receptors, in the absence and presence of streptavidin.

FIG. 8 is a chart comparing the transconductance of a biotinylated, PEI/PEG-coated NTFET device, in the absence and presence of streptavidin that has been complexated with biotin, thereby blocking its binding sites.

FIG. 9A is a model of an exemplary cell membrane (Halobacterium salinarum) for use with a nanostructure electronic sensor for biomolecule detection.

FIG. 9B is a representation of a nanotube network structure for an electronic biomolecule sensor.

FIG. 9C is a schematic diagram showing three orientation cases of exemplary cell membranes: mixed orientation, cytoplasmic side attached, extracellular side attached.

FIG. 9D is a representation showing an exemplary distribution of a cell membrane over a nanotube network for an electronic sensor.

FIG. 9E is a plot of measured cell membrane thickness across a section shown in FIG. 9D.

FIG. 10A is a plot of the device characteristics of a nanoelectronic sensor device incorporating a cellular membrane material before and after application of cell membrane, in a mixed orientation.

FIG. 10B is a chart illustrating calculation of nanosensor device parameters.

FIG. 10C is a plot of the device characteristics of a nanoelectronic sensor device incorporating a cellular membrane material before and after application of cell membrane, with the cytoplasmic side of the cell membrane attached.

FIG. 10D is a plot of the device characteristics of a nanoelectronic sensor device incorporating a cellular membrane material before and after application of cell membrane, with the extracellular side of the cell membrane attached.

FIG. 11A-11B are diagrams illustrating cell membrane geometry in a nanotube sensor device.

FIG. 12 is a schematic cross section of an exemplary capacitance sensor having aspects of the invention.

FIG. 13A-13B illustrate an example of an exemplary device having aspects of the invention employing enzymatic reactions.

FIG. 14A-14B are diagrams showing exemplary devices having aspects of the invention employing liquid or buffer gating.

FIG. 14C-14D are charts showing exemplary electronic responses of devices having aspects of the invention employing liquid or buffer gating.

FIGS. 15A-15B are charts showing an exemplary changes in device characteristic of an exemplary device having aspects of the invention in the presence of an adsorbed species S, as influenced by electron transfer and potential scattering of charge carriers, respectively.

FIGS. 16A-16B are charts showing an exemplary response of an exemplary device having aspects of the invention in response to NH₃ and NO₂.

FIG. 17A is a diagram of an exemplary virus particle such as may be detected using an exemplary nanoelectronic sensor according to the invention.

FIG. 17B is a graph showing an exemplary response to binding of a virus particle in a sensor having aspects of the invention.

### DETAILED DESCRIPTION OF THE PREFERRED EMBODIMENT

The present invention provides a nanotube sensor to selectively sense biological molecules, that overcomes the limitations of the prior art. These advancements have been demonstrated by a nanotube sensor according to the invention, which has been shown to be selectively sensitive to the well-characterized ligand-receptor binding of biotin-streptavidin.

In general, the invention provides a sensor architecture that allows the detection of protein-protein interactions, and also reduces or eliminates non-specific binding. In an embodiment of the invention, an inherently hydrophobic NT-FET, covered with a polymer coating layer with hydrophilic properties, is used as a transducer. The hydrophilicity of the polymer layer reduces the affinity of nanotubes towards non-specific protein binding, which is favored by a hydrophobic environment. In the exemplary embodiment detailed below, biotin is covalently attached to the polymer. When in use, the attached biotin binds with the complementary protein streptavidin, and the formation of the streptavidin-biotin complex is electronically detectable. The streptavidin-biotin complex may serve as a model system for protein interactions, as it has been extensively studied, and the binding is well understood. However, the invention is not limited thereby.

### A Bio-Receptor Functionalized Nanosensor

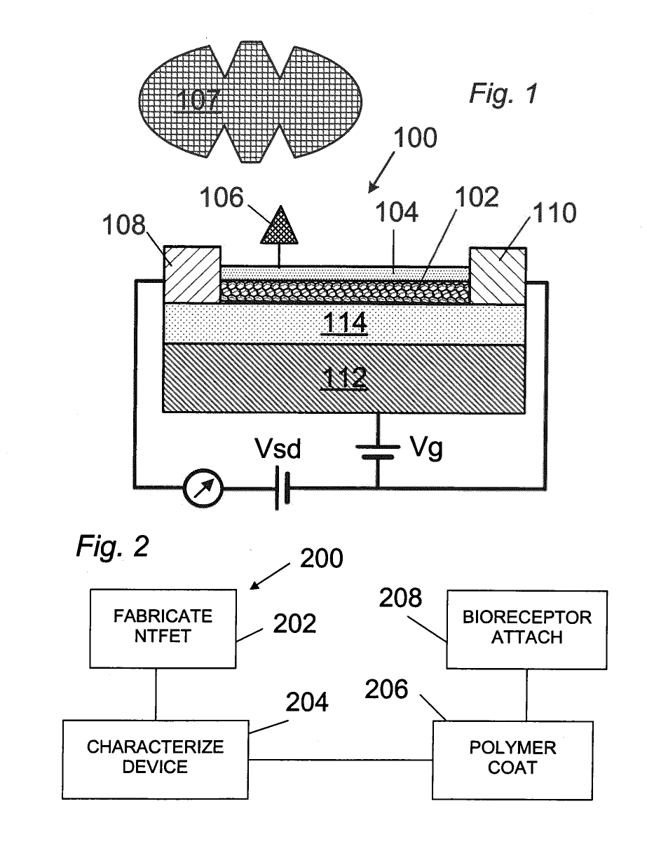

FIG. 1 schematically depicts a sensor 100 that uses a carbon nanotube 102 as a transducer. Nanotube 102 is covered with a polymer coating 104 that has hydrophilic properties and onto which a bioreceptor molecule 106 is attached by a chemical bond to the underlying layer. Bioreceptor 106 may be selected for its selectivity in binding to a biomolecule target 107. Various receptor/target combinations are known, or may be discovered. In an embodiment, the receptor 106 is biotin, and the target 107 is streptavidin. Additional bioreceptor molecules of the same or different types as molecule 106 may additionally be attached to polymer layer 104. A plurality of such bioreceptor molecules (not shown) may disposed over the surface of the polymer layer. The nanotube 102 may be connected to a source electrode 108 and a drain electrode 110 on gate 112. A passivation layer 114 as known in the art, such as SiO₂, may cover the gate substrate 112, which may comprise a silicon or other suitable material.

Functionalization via polymer layer 104 in this sensor architecture has several advantages. First, the polymer is used to attach molecular receptor molecules to the sidewalls of nanotubes, thereby avoiding covalent chemical attachment of biological molecules to nanotubes. Second, polymer coatings have been shown to modify the characteristics of nanotube FET devices, and thus the coating process can be readily monitored. In particular, coating NTFETs with polyethylene imine (PEI) polymer advantageously shifts the device characteristic from p- to n-type. Third, the polymer coating may be useful for preventing nonspecific binding of proteins.

The effect of polymer coating, attachment of a bioreceptor, and subsequent capture of a biomolecule by the bioreceptor on the transconductance of a sensor device according to the invention may be understood as follows, although the invention is not limited thereby. Coating with poly(ethylene imine) (PEI) leads to n-type doping, due to the electron-donating NH₂ groups. PEI is but one example of a polymeric compound that can be utilized in such a way; other examples include poly(ethanol amine) as well as poly(ethylene glycol) (PEG) and polytetrahydrofurane bis(3-aminopropyl)-terminated polymer. Attachment of a bioreceptor, such as biotin, to PEI is through covalent binding to the primary NH₂ group, which would be expected to reduce the overall electron donating function of PEI and cause a transconductance profile that is consistent with indicating removal of electrons from the device. As only the primary NH₂ sites are involved in binding to biotin, the p-type conductance observed before coating is not fully recovered. It is reasonable to postulate that upon streptavidin-biotin binding, geometric changes occur which locally perturb the coating, thereby reducing the effectiveness of the charge transfer and altering the transconductance of the device. It is worth noting that functionalization via the primary NH₂ group of the PEI or other polymer layer could be applied to oligonucleotides, as well as to proteins.

Besides providing desirable electrical properties, layer 104, due to its hydrophilic qualities, may reduce the affinity of nanotubes toward protein binding and thereby improve the selectivity of the device. A variety of polymer coatings and self-assembled mono-molecular layers have been used to prevent binding of undesired species on surfaces for biosensor and biomedical device applications, and may also be suitable for use with the invention. Among the various available polymers for coating, poly(ethylene glycol) is one of the most effective and widely used.

An exemplary method 200 for fabricating FET devices like device 100 with nanotubes as the conducting channel is diagrammed in FIG. 2. At step 202, a p-type NTFET may be fabricated using nanotubes grown by chemical vapor deposition (CVD) on 200 nm of silicon dioxide on doped silicon from iron nanoparticles with methane/hydrogen gas mixture at 900 deg C. Electrical leads may be patterned on top of the nanotubes from titanium films 35 nm thick capped with gold layers 5 nm thick, with a gap of 0.5 to 0.75 μm between source and drain. Multiple nanotubes may be connected to the source and drain electrodes, with the individual tubes varying from metallic to semiconducting. Consequently, a range of device modulations (expressed as the ratio of the “on” to the “off” source-drain current, measured at −p10 V and +10 V gate voltage, respectively) may be observed. Such devices will display p-type transistor behavior prior to functionalization with a suitable polymer layer. Exemplary devices resulting from the foregoing process may have 0.5 μm wide pairs of electrical leads separated by 0.5 to 0.75 μm gaps, and these gaps may be bridged by 1 to about 5 nanotubes along a 10 μm length of a pair of leads. It should be apparent that numerous other configurations may also be suitable.

At step 204, the device characteristic for the NTFET may be determined. As used herein, “device characteristic” refers to the dependence of the source-drain current, I_{sd}, which is a function of the gate voltage V_{g}, I_{sd} (V_{g}), measured from +10 V to −10 V. Any other suitable measure may also be used to characterize the NTFET device. The device characteristic may be used later as a baseline for subsequent calibration of the device's electrical response.

It should be noted that the NTFET examples shown may be usefully employed with other measurement schemes, such as a resistance sensor at zero gate bias, and as a capacitance sensor measuring capacitance, impedance and/or like properties of the nanostructure elements (e.g. carbon nanotube network) relative to the gate electrode or another reference electrode.

After determining the device characteristic, a polymer functionalization layer may be deposited over the device at step 206. For example, the device may be submerged in a 10 wt % solution of poly(ethylene imine) (PEI, average molecular weight of about 25000, Aldrich) and poly(ethylene glycol) (PEG, average molecular weight of about 10000, Aldrich) in water overnight, followed by thorough rinsing with water. Commercial polyethyleneimine (PEI) may be used; this form is highly branched, has a molecular weight of about 25000, and contains about 500 monomer residues. About 25% of the amino groups of PEI are primary with about 50% secondary, and 25% tertiary. After the coating process, a thin layer (for example, <10 nm) of polymer material should coat the devices. The finished polymer coating may be observed by atomic force microscopy.

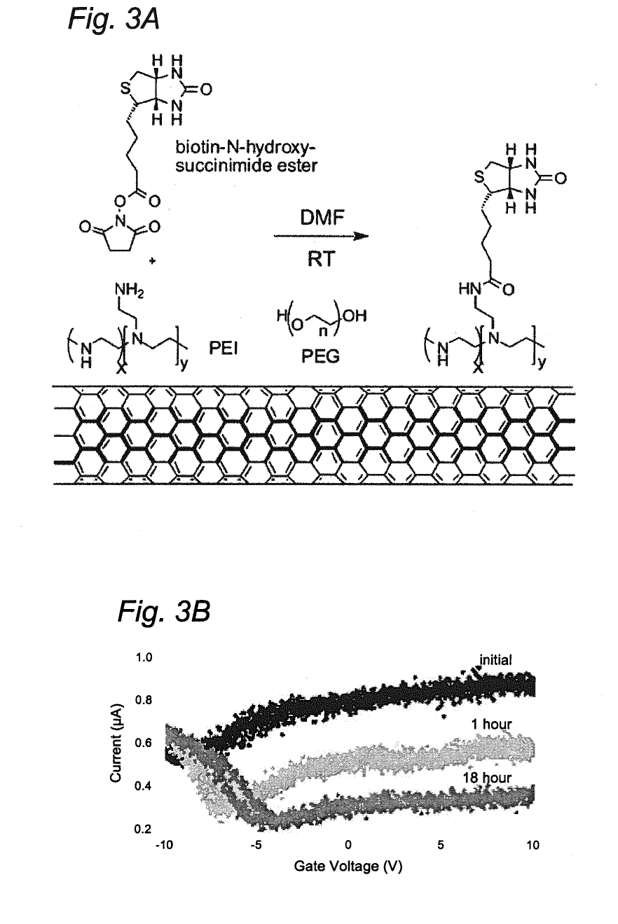

At step 208, the desired biomolecular receptor may be bonded to the polymer layer. If biotin is the desired receptor, a polymer-coated device may be biotinylated by submerging in a 15 mM DMF solution of biotin-N-hydroxysuccinimide ester (Sigma) at room temperature. This compound readily reacts with primary amines in PEI under ambient conditions, leading to changes of the device characteristic as will be discussed below. After soaking overnight, devices may be removed from solutions rinsed with DMF and deionized water, blown dry in nitrogen flow, and dried in a vacuum. FIG. 3A depicts a chemical scheme by which biotin may be attached to the polymer coating. FIG. 3B shows an exemplary transconductance curve for a PEI-coated device prior to the biotinylating reaction, and after 1 hour and 18 hours, respectively, of the reaction.

The device characteristics may be examined after drying, as reported herein. While the device may also exhibit a response in a buffer or other fluid, the examples herein should serve to illustrate the changes of the device characteristic, brought about by different chemical and biological modifications. Such direct correspondence may be somewhat obscured in a buffer environment.

Illustrative results are reported below. After drying, biotinylated polymer-coated devices constructed according to the foregoing description were exposed to a 2.5 μM solution of streptavidin 15 in 0.01 M phosphate buffered saline (pH 7.2, Sigma) at room temperature for 15 min. Subsequently, the devices were thoroughly rinsed with deionized water and blown dry with nitrogen.

An atomic force microscope (AFM) image of one of the devices after exposure to streptavidin labeled with gold nanoparticles indicated the presence of streptavidin. Based on the image, it appeared that streptavidin was effectively attached to the biotinylated PEI polymer coating the nanotubes. The imaged device comprised a nanotube about 800 nm long, and approximately 80 streptavidin molecules were surmised to be in direct interaction with the nanotube conducting channel.

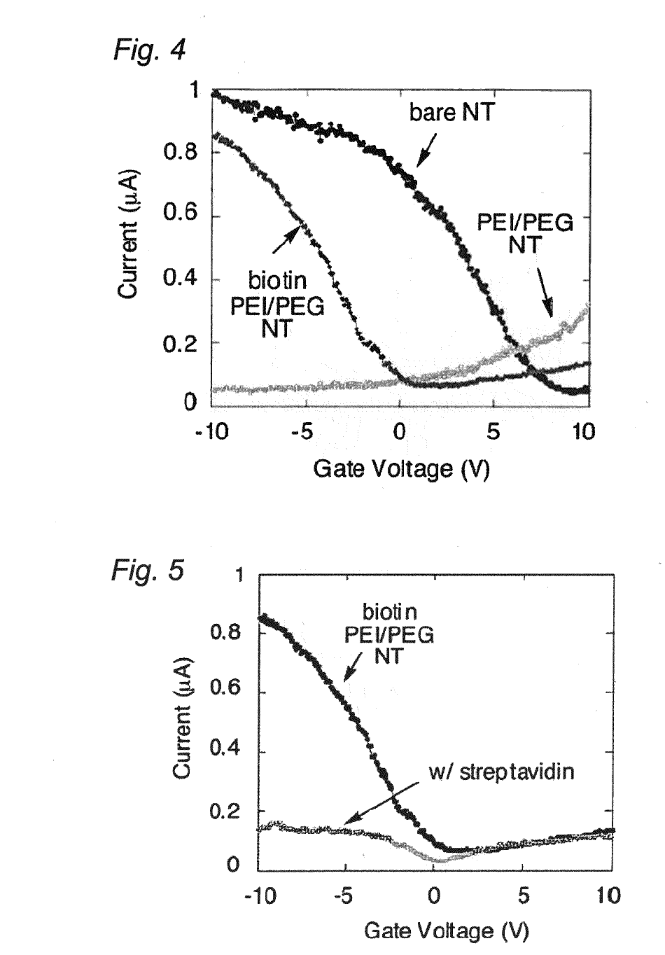

The device characteristic of the sensor before chemical modification was p-type in an ambient environment, presumably due to exposure to oxygen. Coating the device with the mixture of PEI and PEG polymers resulted in an n-type device characteristic, as shown by FIG. 4. The electronic characteristic of the device after 18 hours of biotinylation reaction is also depicted in FIG. 4. Note that the p-type conductance observed before coating with PEI is not fully recovered after functionalization with biotin.

The effect of exposing the biotinylated polymer-coated device to a streptavidin solution and the control experiments (conducted on different devices) is shown in FIG. 5. A striking loss of source-drain current for negative gate voltages after exposure to streptavidin and consequent streptavidin-biotin binding is evident, with little shift of the device characteristic toward negative or positive gate voltage.

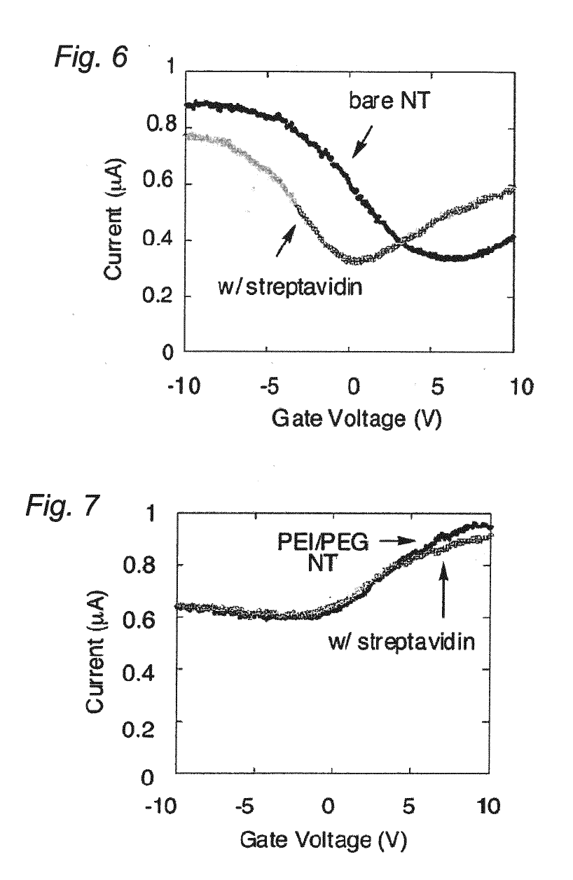

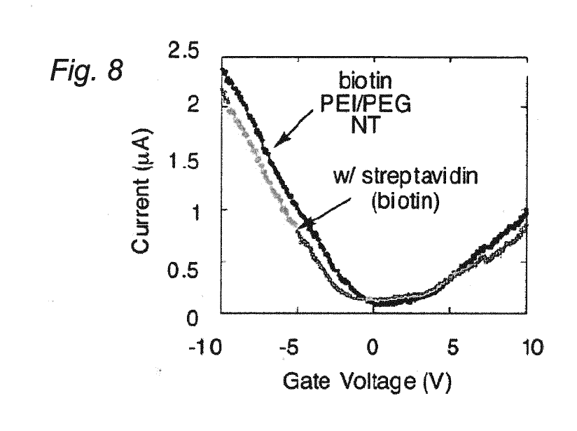

Several control experiments were performed to demonstrate the effectiveness of the device architecture in avoiding false positives and in detecting specific protein binding. First, the uncoated NTFET device was exposed to streptavidin. A change of the device characteristic, as shown in FIG. 6, may indicate attachment of streptavidin to the device. Note, however, that in this case the primary effect is the shift of the device characteristic toward negative gate voltage. In contrast, when the device was polymer-coated, but not biotinylated, no changes occurred upon exposure to streptavidin, as demonstrated by FIG. 7. This suggests the effectiveness of the polymer coating in preventing direct, nonspecific interaction of streptavidin with the nanotube. Finally, addition of a streptavidin in which the biotin-binding sites were blocked by complexation with excess biotin produced essentially no change in device characteristic of the biotinylated polymer-coated device, as demonstrated by FIG. 8.

Several conclusions on the effect of biomolecules on the device electronics may be drawn. First, exposing the bare, uncoated device to streptavidin leads to the shift of the transconductance toward negative gate voltages, thereby rendering the device less p-type, with little reduction in the magnitude of the transconductance. This indicates that the primary effect of the nanotube-streptavidin binding is a charge-transfer reaction with streptavidin donating electrons to the nanotube. Biotin-streptavidin binding has a different effect; in this case the current is reduced. At the same time the device characteristic is modified only for negative gate voltages as shown by FIG. 5, leaving the transcoductance in the positive gate voltage region unaffected.

Interestingly, similar effects may be observed in devices to which charge carriers were deposited. Such observed effects may be due to localization (delocalization) of positively (negatively) charged ionic entities by a negatively (positively) charged surface. Such a mechanism may also be effective with the disclosed nanotube device, and the mechanism may open the way for electronic modification of bioreactions.

With improvements in NTFET devices, they may also be rendered sensitive enough that single protein detection and monitoring can be achieved. As can be inferred from FIG. 5, the total change in transconductance exceeds the noise level by a factor of about 10. According to the AFM image of a device described above, there are approximately 100 protein molecules in close proximity to the carbon nanotube. Combining these two numbers, our current detection level is estimated to be of the order of 10 streptavidin molecules.

Similar detection sensitivity can be inferred from experiments we have conducted on uncoated nanotubes incubated with streptavidin, for which illustrative results are shown in FIG. 6. This is in contrast to the relatively modest change observed in devices where the active element is a nanowire—a channel with a substantially larger cross section.

Thus, label-free electronic sensing with a nanotube based transducer as the central sensor element may provide other significantly useful features in the detection of biological molecules. Such sensors are small, fast, require very little power, and thus generate little heat. The active sensing area is sized for individual proteins or viruses, and small sample volume in general, and is extremely sensitive as all the current passes through the detection point. Importantly, devices can be made specific to individual molecules, and potentially their response to different molecules can be controlled by using chemical and biological functionalization. Direct detection of specific oligonucleotides, in some ways, is typically even more challenging, and thus represents information more valuable than that of detecting individual proteins. Oligonucleotides in a sample generally show a high degree of variation, based on sequence, and often species of particular interest are rare from two perspectives, as a sample can contain populations of many oligonucleotide species very similar to the ones of interest, and at much higher concentrations.

### Integration of Cell Membranes and Nanotube Network Devices

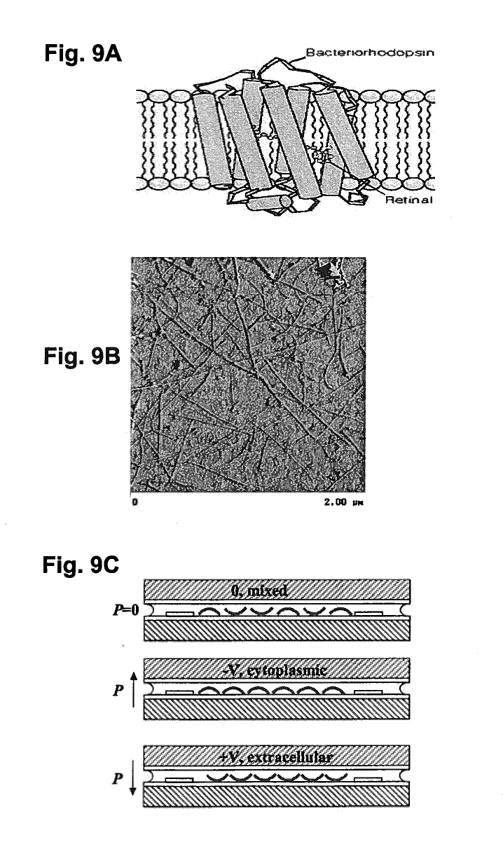

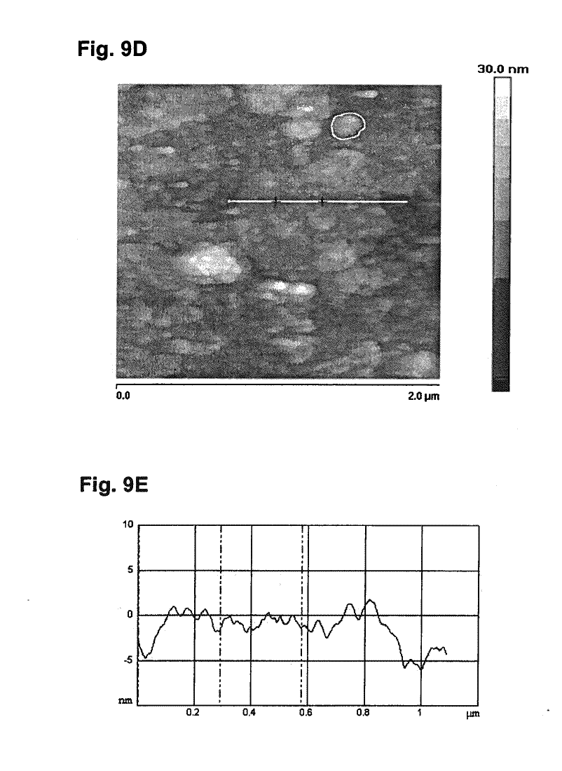

Exemplary embodiment of nanoelectronic devices having aspects of the invention include the integration of a complex biological system and a nanoelectronic device, demonstrating that both components retain their functionality while interacting with each other. Various biological systems may be suitable, including, for example, cellular membranes (walls). In nature, one function of cellular walls is to selectively transfer biological molecules in or out of the cell. This and other aspects of cellular membranes may be used in conjunction with a nanoelectronic sensor to provide a electronic sensor that selectively responds to biomolecules. Cell membranes from numerous sources are believed to be useful in a nanoelectronic sensor, for example, from unicellular and multi-cellular organisms, including both plants and animals. The characteristics of the cell membrane may vary according to organism and cell type, and a suitable membrane may be selected from a diverse array of possible sources. In an embodiment of the invention, a cell membrane Halobacterium salinarum, shown modeled in FIG. 9A, was used to construct a nanoelectronic sensor.

In an embodiment of the invention, the nanoelectronic device may be configured generally similar to that of FIG. 1, which includes a nanotube network transistor. The nanotube device may comprise a random carbon nanotube network generally of as shown in FIG. 9B. FIG. 9B represents an image (amplitude signal) of a exemplary nanotube network such as may be obtained by atomic force microscopy. The nanotube network may incorporate many individual nanotubes in such a way that entire patches of cell membrane are contacted by nanotubes. In this embodiment, the network contains many randomly oriented carbon nanotubes, grown on a substrate (silicon oxide on metallic silicon) by CVD. Nanotube networks may also be formed by alternative methods, such as solution deposition, vacuum filtration, and the like. The nanotubes generally occur individually, rather than in bundles.

In this embodiment, the density is adjusted so that the network functions as a transistor when influenced by a bias voltage from a gate electrode, such as a gate voltage applied to the buried conductive substrate. Embodiments of devices having aspects of the invention include patches of cell membrane covered a dense network of individual carbon nanotubes contacted by metal electrodes (see Bradley et al., Flexible Nanotube Electronics, Nano Letters (2003) 3, 1353-55; and U.S. patent application Ser. No. 10/846,072, filed May 14, 2004, entitled “Flexible nanotube transistors” which is incorporated by reference), referred to as a nanotube network field-effect transistor (NTN-FET).

According to this embodiment, the biophysical properties of the membrane are preserved and the nanoelectronic device functions according to its electronic design (e.g, as transistors, capacitors and the like) when integrated with the membrane. The two systems (biological and electrical) should interact to produce measurable effects, useful for a range of industrial, scientific and medical purposes, such as biological or medical sensing and detection, electro-biological control or data acquisition systems, artificial neuro-sensory organs, and the like. Further, the interaction may be used to determine the charge distribution in a biological system, e.g., so as to permit a bioelectronic device to be optimally configured without undue experimentation. For example, by means of an exemplary embodiment, it was determined that the electric dipole of the example membrane protein bacteriorhodopsin is located ⅔ of the way from the extracellular to the cytoplasmic side.

Nanobioelectronics, the integration of biological processes and molecules with nanoscale fabricated structures, offers the potential for electronic control and sensing of biological systems. As a specific example, carbon nanotubes have been suggested for use as prosthetic nervous implants in organs such as eyes and ears. To achieve this goal requires the parallel preparation of fully functional biological systems and nanoelectronic systems that are integrated together. One major obstacle is the preservation of functionality in both systems. For example, while biological systems ranging from lipids to living cells have been assembled on nanotube substrates, the nanotubes have served only as mechanical supports, without electronic functionality. A second major obstacle is the difference in scale between nanostructures and biological systems. While nanotubes are comparable in size to individual proteins, they are much smaller than cells. Thus, it has previously been attempted to use nanotube electronic devices as single-molecule sensors rather than to communicate with complex biological systems. According to this embodiment of the invention, however, nanoelectronic devices achieve integration between a functioning nanotube transistor and a cell membrane.

Nanotube networks, a recently developed class of nanotube devices, are useful to bridge the gap in size between nanotechnology and biotechnology. This is shown to be a powerful approach, permitting composite nanoelectronic/biological devices to extract information about the charge distribution in the particular membrane used, thereby contributing to the resolution of a long-standing question about charge distributions within that membrane.

Use of a cell membrane disposed against a nanotube network or equivalent nanostructure can provide several significant features and advantages. First, the cell membrane is in direct contact with the semiconducting channel of the transistor. This is distinct from previous work, in which cell membranes have contacted the gate electrodes of transistors, and the transistors detect the electrical potential across membranes. In contrast, in the example of FIG. 9A-E, the devices detect local electrostatic charges on the biomolecules. This is possible because the nanotubes are robust, air-stable semiconductors that can be exposed to cell membranes.

Second, the use of a large number of nanotubes ensures that entire patches of membrane are in contact with nanotubes. Thus, the size scale of nanotechnology, which enables the semiconductor integration, is interfaced with the larger size scale of biology. Note that the preservation of transistor operation in a device with many nanotubes requires careful control of growth parameters (as described in Bradley et al., above), because metallic nanotubes will otherwise shunt much of the transistor current.

One exemplary embodiment having aspects of the invention includes a portion of purple cell membrane (PM) of from Halobacterium salinarum, an organism which has been widely studied. PM contains the light-sensitive membrane protein bacteriorhodopsin, which serves as a photochemical proton pump and has been used to fabricate phototransistors. In addition, rhodopsin has a permanent electric dipole moment, a charge distribution which produces an electric field pointing from the extracellular side of the membrane towards the cytoplasmic side. These properties make PM an ideal prototype membrane for nanobioelectronic integration.

In one aspect, the dipole is employed as an indicator that the integration preserves the biomaterial while bringing it into contact with the nanoelectronic devices. In another aspect, the dipole moment of the PM (or an alternative cellular or quasi-cellular component having a dipole) is employed to electrically influence the properties of adjacent nanostructures included in an exemplary nanoelectronic sensor embodiment having aspects of the invention, so as to produce measurable changes when the membrane interacts with a target species, such as an analyte of interest.

For example, in a carbon nanotube capacitance sensor embodiment, the dipole moment of the PM may serve to increase the effective capacitance of the sensor, so that interactions of the PM with species which cause the dipole moment of the PM to change are in turn detected by the sensor as a measurable change in sensor capacitance. For example, an analyte of interest may absorb onto or intercalate into the membrane so as to cause the dipole to change.

As shown in FIG. 9C, in certain embodiments having aspects of the invention, PM isolated from Halobacterium salinarum may be deposited on previously fabricated NTN-FETs (see section “Methods of Making Nanobioelectronic Devices.” below). To determine the effect of the electric dipoles fixed in the PM, devices may be prepared in three conditions: with the cytoplasmic side of the PM facing the nanotubes, with the extracellular side facing the nanotubes, or with a mixture of both orientations.

FIG. 9C includes three views which show the alternative arrangements of deposition of PM. A silicon chip with a thin oxide coating may be placed on top of a sample chip, leaving a few microns of liquid between the substrate having the nanoelectronic device and the covering plate. Three deposition conditions were included:

The top level (P=0) shows a schematic of the device integrated with the cell membranes such that the orientation of the intracellular and extracellular surfaces of the membranes is generally random, resulting in approximately equal areas of CNT network contacted by cytoplasmic and extracellular surfaces of the membranes. In the top portion, called mixed-orientation, the top chip has zero electrical potential, so that the rhodopsin dipoles point up and down with equal frequency. As a result, the PM contacts the nanotubes with both sides, and the net dipole moment ‘P’ is zero.

The middle level (P↑) shows the device integrated with the cell membranes such that the orientation of the cytoplasmic surfaces of the membranes is generally towards the CNT network. In the middle portion, cytoplasmic orientation is shown, with −3 V on the top chip, so that the net dipole moment is upwards.

The bottom level (P↓) shows the device integrated with the cell membranes such that the orientation of the extracellular surfaces of the membranes is generally towards the CNT network. In the bottom portion, extracellular orientation is shown, with +3 V on the top chip, so that the net dipole moment is downwards.

FIG. 9D represents a topograph of a completed nanobioelectronic device with mixed-orientation PM coating a nanotube network, such as may be obtained using atomic force microscopy. PM is visible as irregular patches, one of which is outlined at upper right. The vertical bar at right displays a key for greyscale coding indicating the thickness of PM coating. The horizontal white line towards the center of the image indicates the contour selected for a line section of the topographic data, shown in FIG. 9E.

As shown in FIG. 9E, the PM patch along the selected contour is uniformly about 5 nm high. In an embodiment of the invention prepared according to the foregoing, the cellular membrane films were measured by AFM to be 5 nm thick, indicating a monolayer PM deposition. Before and after deposition of PM, the NTN-FET transfer characteristics (conductance versus gate voltage) were measured. Other deposition thicknesses, such as multi-layer thicknesses, may also be suitable.

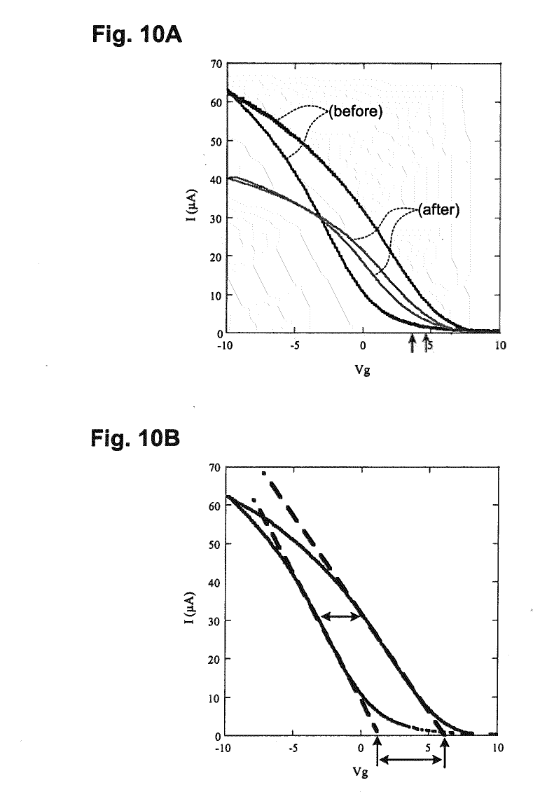

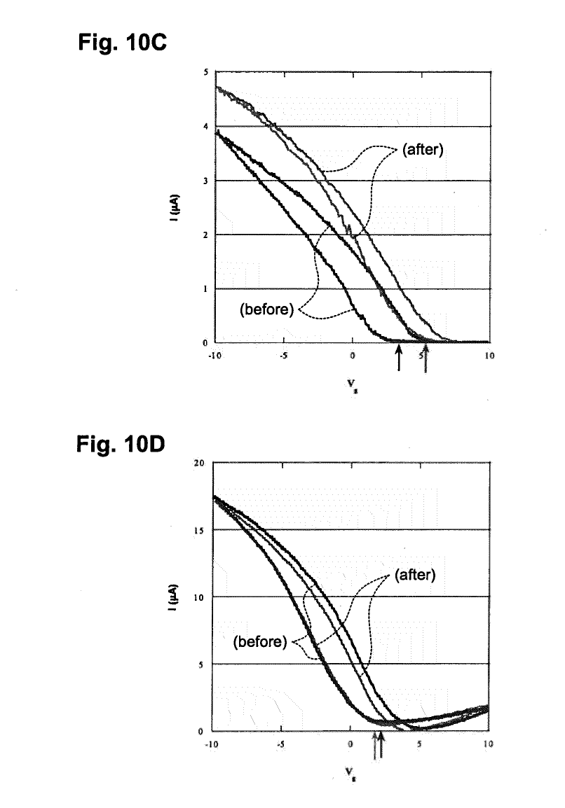

FIG. 10A shows exemplary transfer characteristics of an exemplary device as influenced by the deposition of PM, i.e., after deposition in comparison to before deposition. In this example, the deposition layer comprised mixed-orientation PM. The curves show transfer characteristics (current versus gate voltage) (bias voltage=100 mV) for a device before (black curve) and after (purple curve) the deposition of cell membrane. Each transfer characteristic has two curves, from the right-moving sweep of gate voltage and the left-moving. The intrinsic threshold voltage, indicated by black and purple arrows respectively, is the average between the two sweeps.

FIG. 10B shows a schematic of transfer characteristics, illustrating the calculation of the device parameters for an exemplary device. The width of the hysteresis is indicated by the pairs of horizontal arrows, drawn at a conductance of 50% of the maximum. The transconductances for the right- and left-moving sweeps are shown by dashed lines. Each transconductance is extrapolated back to zero current, where its intersection with the x-axis is the right-moving or left-moving threshold voltage. These two threshold voltages are indicated by arrows on the axis. A midpoint between them may be selected as an intrinsic threshold voltage, in this example at about 3V.

The device embodiments shown in FIGS. 10A and 10B operate as p-type transistors, conducting well at negative gate voltages and not conducting at positive gate voltages. In the region of zero gate voltage, the devices turned on sharply as the gate voltage was changed; this sharp turn-on, or high transconductance, has been attributed to the high mobility of charge carriers in carbon nanotubes. The sharp turn-on begins at a specific gate voltage, referred to as a threshold voltage. However, the devices showed significant hysteresis, in that different threshold voltages were measured using left-moving and right-moving sweeps of the gate voltage. The intrinsic threshold voltage is taken to be the average between the left-moving and right-moving threshold voltages.

FIG. 10A highlights three main device parameters before and after deposition of a mixed-orientation cellular membrane layer for an exemplary device. The changes described here were observed repeatedly in several devices prepared in the same way. First, the hysteresis loops narrowed significantly, as indicated by the arrows. In this case, the width decreased from 3.5 V to 0.8 V. Second, the threshold voltage changed by +1.0±0.2 V, as indicated by the arrows on the x-axis. Finally, the transconductance decreased by about 20%. As discussed below, these changes show that the PM has been successfully integrated with the NTN-FETs.

FIGS. 10C and 10D show the characteristics and effects of oriented PM deposition. In both orientations, the membrane deposition caused a narrowing of the hysteresis loops similar to that caused by the mixed-orientation deposition. At the same time, the threshold voltages shifted, in opposite directions according to the orientation of the membrane. Note that the transconductance did not change, although the maximum conductance changed in accordance with the shifts in the threshold voltage.

FIG. 10C shows the transfer characteristics (bias voltage=100 mV) before (black) and after (purple) the deposition of membrane oriented with the cytoplasmic side contacting the nanotubes. For the cytoplasmic orientation, the hysteresis width decreased from 1.2 V to 0.8 V; and the threshold voltage shifted by +2.2±0.2 V.

FIG. 10D shows the transfer characteristics (bias voltage=100 mV) before (black) and after (purple) the deposition of membrane oriented with the extracellular side contacting the nanotubes. For the extracellular orientation, the hysteresis width decreased from 1.3 V to 0.9 V; and the threshold voltage shifted by −0.4±0.2 V.

Thus, while the invention is not limited thereby, a number of features of the integrated biological/nanoelectronic devices are demonstrated in FIGS. 10A-10D: First, the transconductance of a nanobioelectronic transistor is shown. This quantity is associated with the capacitance between the nanotube network, which forms the channel of the NTN-FET, and the gate; and with the mobility of carriers within the nanotube network. The gate-network capacitance is shown to be constant as a result of membrane deposition; this is confirmed by the fact that the transconductance is not changed by oriented membrane deposition (FIGS. 10C, 10D). In the case of mixed-oriented membrane deposition, the alternation of positive and negative electric dipoles on a length scale of about 500 nm (the diameter of a typical patch of PM) acts as a significant random scattering potential, which decreases the carrier mobility in the network. Thus, the decrease in transconductance in FIG. 10A is a direct result of the mixture of orientations.

Second, the hysteresis decreased significantly in all cases as a result of the biological coating. The hysteresis results from adsorbed water on the substrate; in addition, coatings which displace water from the nanotubes reduce the hysteresis. Consequently, there is a decrease in hysteresis here as well, as the PM remains intact as a layer contacting the nanotubes. Moreover, the width of the remaining hysteresis is similar for all three conditions, which indicates that the amount of PM coverage is similar. This conclusion was confirmed in randomly selected spots that were imaged by AFM.

Third, the shift of the threshold voltage in the devices results from the electrostatic field associated with the bacteriorhodopsin electric dipole. This field induces charge in the nanotubes, thus shifting the Fermi level. The position of the Fermi level is measured by the threshold voltage, and there is an relationship between the threshold voltage in various device configurations and the quantity of charge induced in the nanotubes. In this example, with a typical nanotube diameter of 2 nm, every 1 μm of nanotube length has a capacitance to the gate, C_{bg}, of about 15 aF. The induced charge, ΔQ, is given by ΔQ=C_{bg}ΔV, where ΔV is the threshold shift. Thus, the +1.1 V shift caused by mixed-orientation PM deposition corresponds to an induced charge of 16 aC/μm of nanotube length. Note that this dipole effect is important to the second embodiment type of this example, the nanoelectronic capacitance sensor.

Thus, by demonstrating these three device parameters, it is shown that the nanobioelectronics integration is successful. First, the NTN-FETs' transistor functionality is preserved. Second, the PM remains intact as a layer, and the bacteriorhodopsin membrane proteins retain their electric dipoles. Third, the deposited PM is demonstrated to contact the NTN-FETs directly and to interact with their electrical properties.

The examples of FIGS. 10A-D demonstrate a significant asymmetry between cytoplasmic and extracellular orientations. This asymmetry is reflected in the large amount of charge induced in mixed-orientation devices, since without an asymmetry, the charge induced by equal amounts of cytoplasmic- and extracellular-oriented PM would cancel. Observations indicate that the mixed-orientation film contains equal amounts of cytoplasmic and extracellular orientations. First, it is shown that our deposition method produces similar coverages for both orientations. Therefore, neither orientation adsorbs preferentially compared to the other, and a random mixture should contain equal amounts of each. Second, the threshold shift observed with mixed orientation correlates with the expectation from a 50%-50% mixture. The two oriented depositions cause +2.2 V and −0.4 V of threshold shift. For a 50%-50% mixture, we expect a net threshold shift of ½(2.2-0.4)V, or +0.9 V. This value agrees well with the value observed with mixed orientation, 1.0±0.2 V. From these two observations, we conclude that the mixed-orientation film is in fact a 50%-50% mixture.

Such an asymmetry results from the fact that the dipole is closer to one side of the PM than the other. Here we are able to observe this asymmetry directly because of the device configuration in which the PM contacts the nanotubes directly.

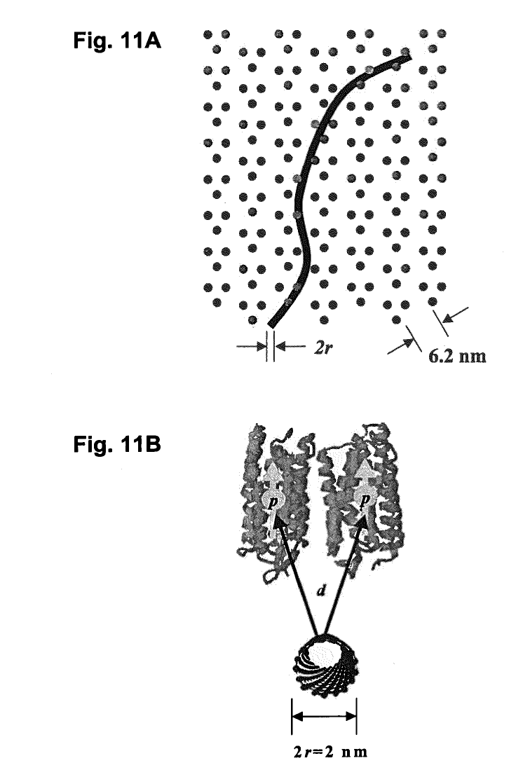

Purely for illustrative purposes, and not by way of limitation, FIGS. 11A and 11B illustrate conceptual modeling of the electrostatic effect of the bacteriorhodopsin dipole on the nanotubes, permitting quantification of the asymmetry. In this regard, the background charge due to the phosphate heads of the lipids of the PM is 0.2 electrons per square nanometer, which is too weak to explain the charge induced in the example devices. The rhodopsin dipole is known to result from the competition between several charge distributions that result in a net dipole moment of 3.3×10⁻²⁸ C·m per rhodopsin monomer.

FIG. 11A illustrates an electrostatic conceptual model of the geometry of the PM and rhodopsin molecules with respect to the nanotubes, which may be used to calculate the effect of this dipole on the nanotubes. The rhodopsin molecules are shown above a nanotube and form a line of constant dipole density, as further discussed in the section “Model of An Integrated nanobioelectronic device”, below. Rhodopsin (purple dots) assembles into trimers, which are arranged on a hexagonal lattice. Each nanotube resembles a curved line which meanders across the lattice, contacting rhodopsins over its width of about 2 nm. Since the rhodopsin dipole density is about 6.0×10⁻²⁹C·m/nm, the nanotube contacts a line density of π=1.2×10⁻²⁵ Cm/μm along the nanotube length.

FIG. 11B illustrates a detailed conceptual model of the association of rhodopsin molecules with a nanotube, and illustrates the dimensions used in the calculations. A typical nanotube has a typical diameter of about 2 nm. A rhodopsin monomer situated near a nanotube has a dipole moment ‘P’. Although this dipole arises from a complex extended charge distribution, it is represented by a point dipole for simplicity. This point dipole is situated within the rhodopsin at a distance ‘d’ from the nanotube surface. In the model, the line of dipoles with a density π induces a charge density, λ, given by λ=−π/d² . Thus, by combining the known dipole moment of bacteriorhodopsin with the induced charge (measured from the threshold voltage shift and the known capacitance), we calculate how far the dipoles lie from the nanotubes.

The answer will be different for the two different orientations, reflecting the position of the dipoles closer to one side of the PM. For the cytoplasmic orientation, with ΔV_{cp}=+2.2V, we calculate d_{cp}=1.9 nm. For the extracellular orientation, with ΔV_{ec}=−0.4V, we have d_{ec}=4.4 nm. Since the sum of these distances, 6.3 nm, is comparable to the membrane bilayer thickness of 5 nm, we conclude that this simple model is reasonable. Note, in particular, that since the ratio between ΔV_{cp} and ΔV_{ec} is 5.5, the electrostatic model indicates that d_{cp} is 2.3 times smaller than d_{ec}. Thus, measured results from exemplary embodiments may provide additional details about the asymmetry of the bacteriorhodopsin charge distribution.

The devices and measurements in the foregoing section demonstrate the integration of nanoelectronic devices, such as carbon nanotube transistors, with biological structures, so as to provide a useful nanobioelectronic system. As a result, a mechanism is provided to connect living cells directly to these nanoelectronic devices.

### Methods of Making Nanobioelectronic Sensor Devices.

Nanotube network transistors (NTN-FETs) were fabricated as described previously. Degenerately doped 100 mm silicon wafers with 200 nm thermal oxide coatings were coated with iron catalyst, and single- and double-walled carbon nanotubes were grown by chemical vapor deposition. The resulting films contained individual nanotubes dispersed over the substrate, contacting each other in a tangled network. Titanium/gold leads were deposited to serve as source and drain contacts with 50 μm separation. At this separation, the source-drain conductance is determined by the nanotube channels, rather than by Schottky barriers at the contacts. After contact deposition, the device region was defined, by removing the nanotubes outside a defined area by oxygen plasma etching.

Purple membrane (PM) was isolated from Halobacterium salinarum, and a suspension of PM in water was prepared at a rhodopsin concentration of 1 mM. Before coating the NTN-FETs, the suspension was freshly mixed with a shaker and warmed to 27° C. A drop of suspension was placed on a chip, and the chip was covered with a blank piece of silicon substrate. The assembly was kept in a chamber at 50% RH for 5 minutes, after which the NTN-FET was blown dry. This procedure was repeated three times to produce films of mixed-orientation PM coating the nanotube network.

The film thicknesses were measured by AFM to be 5 nm, which corresponds to monolayers of PM. To produce oriented films, a voltage of ±3 V was applied between the two chips while they were exposed to the suspension. After the deposition of the membranes, the devices were air-dried for several hours at 40% RH. Electrical properties were measured before deposition and after air-drying, by applying a fixed source-drain bias voltage between contacts on the network and measuring the source-drain current as a function of gate voltage. The membrane suspension and the chips were kept in dark enclosures throughout the experiment to ensure that the bacteriorhodopsin was in its dark-adapted state.

### Model of an Integrated Nanobioelectronic Device.

We use a simple electrostatic model in which the rhodopsin molecules above a nanotube form a line of constant dipole density. Those in the rest of the PM (FIG. 11A) are ignored, because for dipoles farther from the nanotube the induced charge decays rapidly with distance. In this model, the nanotube is considered to be a conducting cylinder of radius r (for our nanotubes, this is typically 1 nm). A length of 1 μm of such a nanotube contacts a membrane area of 2,000 nm². From the known area density of the dipole moment in PM, 6.0×10⁻²⁹ C·m/nm², we calculate that the nanotube contacts a dipole density of π=1.2×10⁻²⁵ Cm/μm. The line of dipoles induces a charge density λ in the conducting cylinder λ=−rπ/d², where ‘d’ is the distance between the nanotube surface and the dipole.

Let us suppose that the rhodopsin dipole is a point dipole embedded within the PM at a distance d_{cp} from the cytoplasmic side and d_{ec} from the extracellular side, as illustrated in FIG. 11. Then the dipole will induce different amounts of charge, λ_{cp} and λ_{ec}, depending on which side contacts a nanotube. For the cytoplasmic case, with ΔV_{cp}=+2.2 V, we have λ_{cp}=33 aC/μm. Using the equation above, we calculate d_{cp}=1.9 nm. Similarly, for the extracellular case, with ΔV_{ec}=−0.4V, we have λ_{ec}=6 aC/μm, and d_{ec}=4.4 nm. Since the sum of these distances, 6.3 nm, is comparable to the membrane bilayer thickness of 5 nm, we conclude that this simple model is reasonable. Note, in particular, that since the ratio between ΔV_{cp} and ΔV_{ec} is 5.5, the electrostatic model predicts that d_{cp} is 2.3 times smaller than d_{ec}. Thus, the charge density of the rhodopsin dipole is situated closer to the cytoplasmic side of the membrane.

### Nanotube Network Capacitive Device Embodiments

In an embodiment of the invention, a nanotube-based capacitance device, e.g., a sensor, may be combined with a biological component generally similar to that described above to provide a composite biological/nanoelectronic device. The principles of bioelectronic integration and the making of nanobioelectronic devices described in connection with the foregoing embodiment are also generally relevant to embodiments of capacitive devices as described below.

Although in the description that follows, the exemplary embodiments are based on one or more carbon nanotubes, it is understood that other nanostructures known in the art may also be employed. Elements based on nanostructures such carbon nanotubes (CNT) have been described for their unique electrical characteristics. Moreover, their sensitivity to environmental changes (charged molecules) can modulate the surface energies of the CNT for use as a sensor or detector. The modulation of the CNT characteristic can be investigated electrically by building devices that incorporate the CNT (or CNT network) as an element of the device. This can be done as a conductive transistor element or as a capacitive gate effect.

Certain exemplary embodiments having aspects of the invention include single-walled carbon nanotubes (SWNTs) as semiconducting or conducting elements. Such elements may comprise single or pluralities of discrete parallel NTs, e.g., in contact or electrically communicating with a device electrode. For many applications, however, it is advantageous to employ semiconducting or conducting elements comprising a generally planar network region of nanotubes (or other nanostructures) substantially randomly distributed adjacent a substrate, conductivity being maintained by interconnections between nanotubes.

Devices fabricated from random networks of SWNTs eliminates the problems of nanotube alignment and assembly, and conductivity variations, while maintaining the sensitivity of individual nanotubes For example, such devices are suitable for large-quantity fabrication on currently on 4-inch silicon wafers, each containing more than 20,000 active devices. These devices can be decorated with specific recognition layers to act as a transducer for the presence of the target analyte. Such networks may be made using chemical vapor deposition (CVD) and traditional lithography, by solvent suspension deposition, vacuum deposition, and the like. See for example, patent application Ser. No. 10/177,929 entitled “Dispersed Growth of Nanotubes on a Substrate”; and U.S. patent application Ser. No. 10/280,265 entitled “Sensitivity Control for Nanotube Sensors”; U.S. patent application Ser. No. 10/846,072 entitled “Flexible Nanotube Transistors,” each of which application is incorporated herein by reference.

The nanoscale elements can be fabricated into arrays of devices on a single chip for multiplex and multiparametric applications See for example, application Ser. No. 10/388,701 entitled “Modification of Selectivity for Sensing for Nanostructure Device Arrays”; application Ser. No. 10/656,898 entitled “Polymer Recognition Layers for Nanostructure Sensor Devices”, application Ser. No. 10/940,324 entitled “Carbon Dioxide Nanoelectronic Sensor”; and Provisional Application No. 60/564,248 entitled “Remotely Communicating, Battery-Powered Nanostructure Sensor Devices”; each of which is incorporated herein by reference.

In contrast to resistive or transconductance measurements that monitor charge transfer and charge mobility, a capacitance responds to the relative ease with which the analyte molecules on the nanotubes may be polarized. A surface capacitance effect may be caused by a large electric field gradient radiating from the nanotubes. Since single wall nanotubes (SWNT) are about 1-2 nm in diameter, field gradients of 108 V/cm can be generated, which is impossible in conventional electrode geometries.

Capacitive sensing may exploit the principle that binding events tend to change the thickness or dielectric properties of the recognition layer, and is therefore dependent on the functionalization of nanotubes; i.e., the properties of the recognition layer. Preferably this layer is very thin and electrically insulating to improve the ratio between capacitance and Faradaic currents. Analyte polarizability can be modulated by peak-peak voltage and the AC frequency providing a 2D image of the analyte for better sensitivity and accuracy. Bode plots may provide the frequency dependence of impedance magnitude and phase angle. Data may be plotted as differential capacitance as a function of time. Capacitance measurements do not require a conduction path and are therefore are flexible in terms of functionalization chemistries.

A CNT network may be included in a capacitive electrode. In an active device, such as a sensor for the detection for bio-analytes, a capacitive electrode may be interrogated with an AC signal. Preferably, a CNT network is integrated with metal electrodes. A CNT network may be included as first charge reservoir or “plate” of a capacitor. A metal electrode may be included as a second plate of a capacitor, or both “plates” may include nanostructure elements. Functionalization on this structure (either on the metal plate, on the CNT network, or on other adjacent elements) allows the biochemical attachment of bio-analytes. See for example, application Ser. No. 10/345,783 entitled “Electronic Sensing of Biological and Chemical Agents Using Functionalized Nanostructures,” and application Ser. No. 10/704,066 entitled “Nanotube-Based Electronic Detection of Biomolecules”, each of which is incorporated herein by reference.

The second plate of the capacitor may include a metallic surface that is separated from the first plate through some dielectric material, in a solid, liquid or gaseous phase, including, for example, air. In an embodiment of the invention, the presence or absence of bioanalytes on the capacitor plate changes the impedance of the structure and can be detected by external measurement equipment. Measurement of capacitance is a well known technique in medical and diagnostic devices. Low cost electronic acquisition chips exist to quantify the change in capacitance (e.g., chips made by Analog Devices, among others).

The change in capacitance can be affected by the dipole moment of the molecules in contact with the capacitor. In addition, large dipole molecules can be included in the system that specifically bind to the analyte of interest (sandwich assay) to further enhance the signal of the detection.

Besides simple analyte binding, the devices can also be used to interrogate cell membranes or cellular events. In particular, it is well known that when bacteriophage disrupt the bacterial membrane, a large ionic gradient occurs. Again, this type of biochemical disruption in the proximity of the CNT capacitance plate can be measured and used as a bacterial species identifier.

Note in this regard the discussion above with respect to PM membranes, in which the electrical properties of the nanotube network changed as a result of the electrostatic field associated with the bacteriorhodopsin electric dipole. This dipole effect is also effects the measured capacitance of exemplary devices including such membranes (and/or other dipole enhancers) as functionalization.

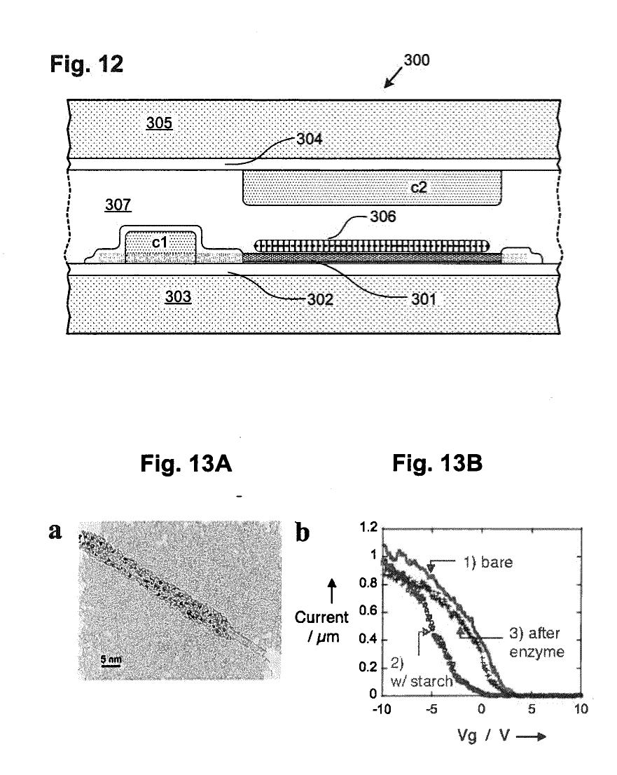

The structure of an exemplary device 300 having aspects of the invention are illustrated in FIG. 12. A nanotube conductive layer 301 (such as a nanotube network) is disposed on the dielectric surface 302 of a lower substrate 303. Contact C1 communicates with the nanotube layer. The contact is shown passivated, as further described in application Ser. No. 10/280,265 entitled “Sensitivity Control for Nanotube Sensors” which application is incorporated herein by reference. A second plate contact C2 may be spaced apart from the nanotube network by an analyte media space 307, and may be disposed on a dielectric surface 304 of an upper substrate 305. As a voltage is applied between C1 and C2, this structure 300 acts as a capacitor. Functionalization may comprise a cell membrane bi-layer 306 applied to the nanotube layer in the manner described herein. The cell membrane responds to at least one analyte of interest in the media 307 so as to produce a measurable change in the capacitance (measurement circuitry not shown).

### Application for Monitoring Enzymatic Reactions

As an example of the application of devices for the electronic monitoring of an enzymatic reaction, monitoring of enzymatic hydrolysis of starch is described below. Starch consists of linear component, amylose which is composed of linkages between D-glucopyranose residues, and amylopectin, the branched one, which in addition to a-1,4 linked D-glucopyranose chains carry branches at C-6 on every 25 or so D-glucopyranose residues which also have the a-configuration. Starch enzymatic hydrolysis may be characterized with amyloglucosidase in acidic buffer, resulting in complete cleavage of the polymer to water-soluble glucose. Enzymatic hydrolysis of starch using amyloglucosidase in solution has been shown to be efficient in precipitating carbon nanotubes from their solution.

Starch-covered single wall nanotubes (SWNT) were studied by transmission electron microscopes. FIG. 13A shows a SWNT covered with starch, as might be seen, for example, using a high resolution electron transmission (HRTEM). For imaging purposes, the starch was contrasted by using RuO₄ staining procedure. After starch deposition, the device characteristic shifts by approximately −2 volts toward negative gate voltages (FIG. 13B) corresponding to electron doping of the nanotube channel by polymer. Compared to other polymers, such as poly(ethylene imine) (PEI), the magnitude of the shift is small. This fact, most likely, relates to difference between electron-donating ability of alcohol and ether groups in starch as compared to amines in PEI. After the enzymatic reaction was completed on the starch functionalized device, the device response observed before starch deposition (FIG. 13B) is recovered, indicating that during enzymatic reaction all the starch is hydrolized to glucose, with the hydrolization product washed off prior to the electronic measurements. Two control experiments were performed to confirm these results. First, the starch functionalized chip was rinsed with buffer to see if the buffer alone can wash away the starch deposited on the device.

The device characteristic after rinsing with buffer solution is similar to that obtained before rinsing, leading to the conclusion that starch removal by buffer alone does not occur. Another control experiment involved the deposition of enzyme solution on bare devices. The device characteristic shows increased hysteresis but no significant shifting has been observed—giving evidence that enzyme alone does not lead to charge transfer.

### Alternative Detection Methods

Liquid Gating.

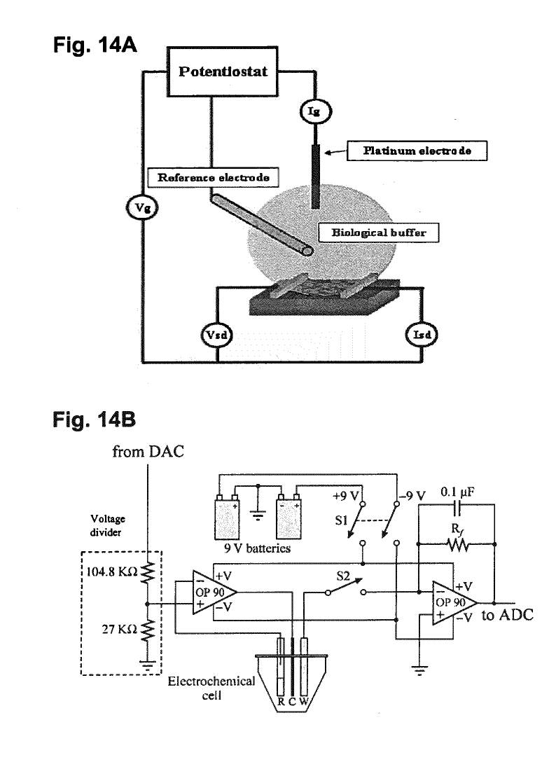

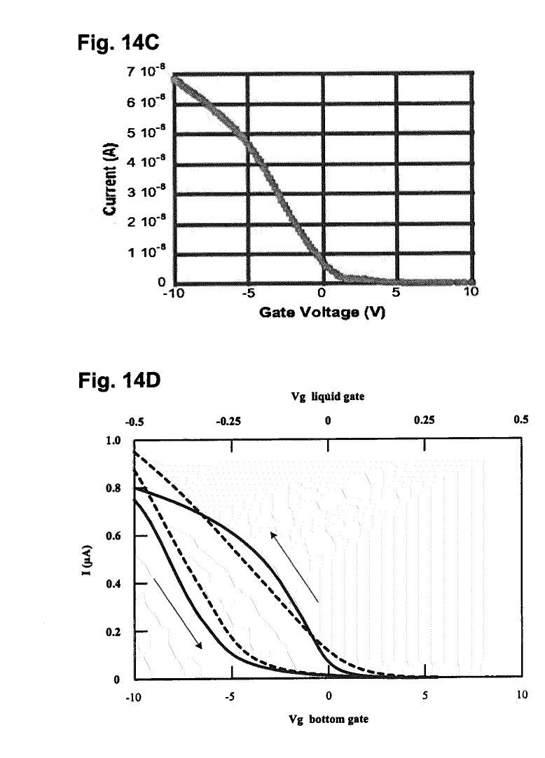

Several alternative detection schemes can be employed for biosensing applications. The presence of an immobilized biomolecule, or the completion of a reaction between biomolecules (such as a ligand-receptor binding for example) can be followed by examining the change of the device characteristics after the biomolecule is immobilized, the reaction completed and the buffer is removed. The device characteristic is measured in a conventional configuration, applying the bottom gate (voltage applied to the substrate, as shown in FIG. 14C). This is appropriate if the mere presence of the biomolecule, or the completion of the biological reaction is examined only, and thus may be an appropriate method for a variety of biotechnology applications.

For some applications, however, It may be preferable to monitor the biological processes that take place in an appropriate buffer environment. Real-time signal acquisition and analysis may have significant impact on the biological sciences for several reasons. First, the time scales for biological processes may be directly measured. The time for a protein to undergo conformational changes, or DNA duplex formation and its complement to form a duplex, could be directly measured. Secondly, the electronic data may lead to seek electronic signatures specific to a biological process. For example, if the binding of different antigens to an antibody each results in a particular electronic signature, then the different antigens may be distinguished from each other. This can dramatically alter the landscape of biological sensing, and aid the development of practical biosensors by solving the problems of false positives and poor cross-sensitivities. Biomolecules undergo a variety of fluctuations and conformational changes that span several orders of magnitude. Picosecond time scales characterize intramolecular vibrations, with an harmonic relaxations on the order of nanosecond. Protein collapse occurs at milliseconds to seconds. The internal time constant of our devices is on the order of microseconds, allowing signal processing at time scales exceeding this limit.

FIGS. 14A and 14B are schematic illustrations of a workstation embodiment having aspects of the invention. This provides for simultaneously measuring electronic and fluorescent signals from molecular binding events that may be used to optimize sensor and assay design without undue experimentation.

The exemplary workstation comprises a 3-electrode electrochemical cell. The reference electrode can monitor the liquid potential between the reference electrode and the working electrode, which is connected to the nanostructure sensor elements, e.g., one or more nanotubes. A small voltage bias is applied to the source-drain electrodes and drain current (Isd) may be monitored as the liquid potential is swept.

A shielded switch box may be used to control which devices are active during operation of the workstation. For example, a PC may be equipped with a National Instruments DAQ card and LabView software to provide data acquisition and a user interface for real time operation. The NTFET devices may be wire bonded and encapsulated in a 40 pin ceramic socket, which may be configured to allow the socket to function on the microscope stage, thus providing simultaneous electronic and fluorescent measurements.

A flow cell may be fabricated to provide liquid delivery for sample application and introduction of wash buffers without unnecessary perturbations. The flow cell minimizes evaporative losses and provides an optical window for fluorescence imaging. Microfluidic channels may be fabricated using standard wet etch protocols and/or PDMS elastomer structures. Fluid delivery may be automated using mechanical or pneumatic pressure driven flow control. Additional capability such as integrated temperature measurement/control and reference electrodes may be included. Note that generally similar structures and components may be included in a disposable sensor cartridge embodiment having aspects of the invention (not shown in FIG. 14).

Fluorescent images may be acquired, for example, using a Zeiss microscope with a TE cooled CCD camera and long working distance objectives. Filter sets for FITC, DAPI, and Cy5 are commercially available. Such images assist may be used verifying that electronic signals are correlated with fluorescent signals without undue experimentation. Electronic modulations produced by bio-recognition events may be detected using conventional test and measurement instruments, such as Keithley source measure units that can detect picoAmps, a Boonton capacitance meter capable of femtoFarads, and a HP impedance analyzer with a 40 Hz to 110 Mhz range.

The fact that a physiological buffer is conducting enables a detection scheme, alternative to “bottom gating.” An electrode is applied to the liquid and I_{sd} is measured as function of the voltage on the electrode, as depicted in FIG. 14C. Several precautions should be made. Electrochemical reactions may take place for large gate voltages, these can be identified (and avoided) by monitoring the current between the gate and the conducting channel. The source and drain electrodes—and all the conducting leads have to be isolated from the buffer in order to avoid non-desirable reactions.

An exemplary device characteristic for both “liquid gating” and “bottom gating” is shown in FIG. 14D. The two configurations result in a similar device characteristic if an appropriate scaling of the x-axis is performed, this scaling is due to the different dielectric layer in the two cases: an oxide insulating layer for bottom gating and a hydration layer in case of “liquid gating”. Monitoring the change of the device characteristic versus time allows the real time monitoring of protein attachment to the device—and a variety of biological processes for that matter.

The transistor configuration is different from usual transistor configurations: here the most sensitive element of the device, the conducting channel, is open to the environment. In addition because of the tubular structure, all the current flows at the surface of the channel are in direct contact with the environment. As the result these devices are extremely sensitive to environmental factors, the presence of different chemical and biological species in the vicinity of the device. The interaction of devices with various inorganic species has been explored in detail, such experiments serve as useful benchmarks for the effects that are observed when the environment is modified. Both exposure to gases and to coating layers have been studied.

Consider a molecule in the vicinity (usually at the surface) of the nanotubes that forms the conducting channel. The effect of such molecule may be similar to the effect of an impurity in a conventional semiconductor with two possible consequences. There may be a charge transfer form the molecule to the nanotube channel, and the molecule may act as a scattering potential.

The results of the two possibilities are different: a charge transfer to the nanotube shifts the device characteristic towards more positive (electron donation form the molecule to the nanotube) or negative (hole donation) gate voltages. In contrast, a molecule may act as a scattering center leading to the decrease of the mobility, thus suppressing the device characteristic without a shift. Such suppression may occur also through a mechanical distortion of the nanotube.

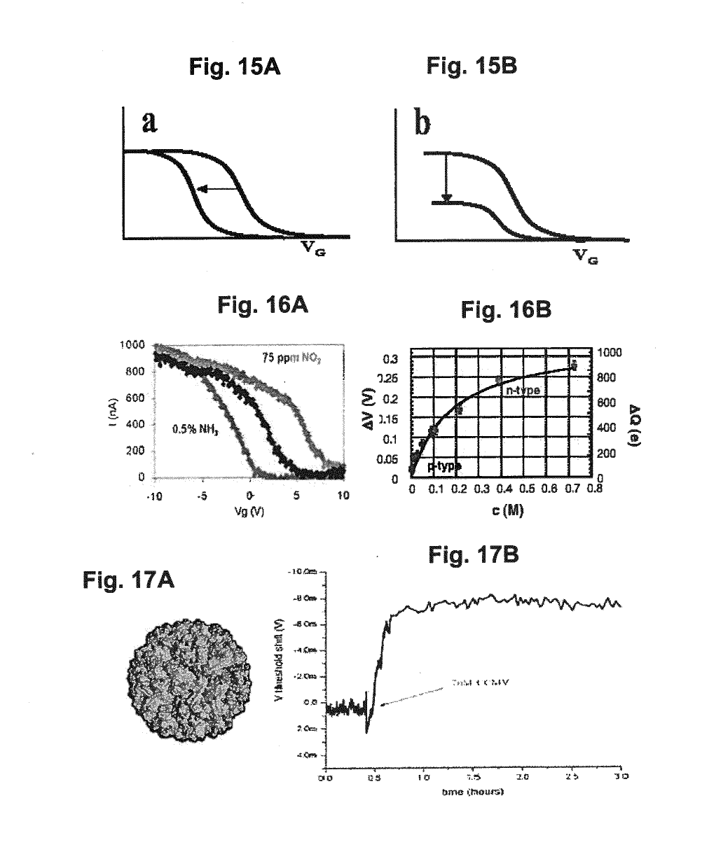

The two situations are depicted in FIGS. 15A and 15B, which show the change of the transistor device characteristic in the presence of an adsorbed species S. FIG. 15A shows electron transfer from S to the nanotube. FIG. 15B shows potential scattering of charge carriers on the potential created by S. Both may occur, and the transistor configuration allows the separation of the two factors, the change of the carrier density and mobility.

Upon exposure to various gases, one finds a shift of the device characteristic, either left or to the right, towards more negative or positive gate voltages, indicating a charge transfer from or to the nanotube, and the effect has been studied in detail. FIG. 16A shows the effect for an electron donating (NH₃) and electron withdrawing (NO₂) species on the transistor device characteristic. Although the notion that the effect is due to the charge transfer between the molecular species and the carbon nanotubes is not universally accepted, unpublished calculations strongly suggest that this is the case.

Gas Interactions.

FIG. 16B shows the shift of the device characteristic, ΔV upon exposure to NH₃ in water, for different ammonia concentrations. The full line describes of what is expected for weak binding of the NH₃ molecules. Under such circumstances the molecules hop on and off the channel, creating a dynamic equilibrium. The coverage of the devices can be calculated as the function of the NH₃ concentration in the liquid, and assuming that the change (in this case the shift of the device characteristic) is proportional to the coverage, the shift can be calculated as function of the concentration. The full line in FIG. 16B represents the calculated dependence—describing to observations to good accuracy. These experiments also confirm that there is a charge transfer from NH₃ to the nanotube channel, with scattering effects playing a lesser role.

Detection of Viruses.

Because of the rich potential of nanobiotechnology, including biosensors and bioelectronics, recent research has focused on the interactions between biomolecules and inorganic systems. A major advantage is the fabrication of structures with proteins immobilized on various functional surfaces, while preserving the biological activity of the proteins. A variety of mechanisms have been explored for immobilization, including covalent bonding, hydrophobic interactions, and charge transfer-induced adsorption. The most direct evidence has been provided by scanning force microscopy, which in recent years has been used to measure the strength of protein attachment. Interactions between biomolecules and various surfaces have been widely utilized, and to some extent studied, however the interaction between the surfaces and the biomolecules are less understood. The interrogation of the device characteristics before and after immobilization offers an opportunity of identifying some of these interactions.

Because of their surface proteins, viruses can also readily interact with the devices and are immobilized. As depicted in FIGS. 17A and 17B, this has been confirmed by experiments using the plant virus CCMV. Just as in case of proteins one observes a shift of the device characteristic indicating charge transfer from the surface proteins to the device. The effect, however is surprisingly small, about an order of magnitude smaller than in case of streptavidin. It appears that binding of the surface proteins to the device is weak, and binding is also hampered by the geometric arrangement of the surface proteins due to their position within the virus structure. Moreover, it is highly virus specific, binding of the virusT4 leads to significantly smaller effect than CCMV. This, by itself is not surprising due to the significant structural and also chemical difference between the two viruses.

The principles and practice of the invention contribute to cell-based electronic sensing: measuring the electronic response of living systems, and to using nanoscale devices for in-vivo applications directed toward cellular physiology, medical screening, and diagnosis. Sensor devices may be constructed, according to the principles of the invention, wherein surface charges can be created on the sensing element when the biological molecules are immobilized, by applying a voltage between elements of the sensor. Such surface charges should interact with the charged bio-molecules, providing further opportunities for selective electronic detection of biomolecules, or electrical manipulation of biological reactions at a molecular level.

The examplary nanoscale electronic devices—e.g., field effect transistors with carbon nanotube conducting channels—interact readily with the environment. For a variety of species, such as reactive gases, polymers with reactive chemical groups and also for proteins and viruses that have been studied our experiments demonstrate that charge transfer occurs between the species and the devices. The change of the device characteristics allows the estimation of the transferred charge for each species. Observations of proteins also suggest strong charge transfer from the protein to the nanotube channel. The charge transfer interaction mechanism identified for proteins has implications on a broad range of areas where immobilization is attempted and used for fundamental studies and also for applications. Such interactions also involve functional groups different from those involved in hydrophobic interactions.

The examples show that one may connect living cells directly to these nanoelectronic devices. As a result, the concepts could be extended to include of what one could call “cellectronics”, cell-based electronic sensing: measuring the electronic response of living systems, and to using nanoscale devices for in-vivo applications: studying cell physiology, medical screening and diagnosis. The sensor architectures can be turned into devices where—by applying a voltage between elements of the sensor—surface charges can be created on the sensing element where the bio-molecules are immobilized. Such surface charges will interact with the charged bio-molecules, but such, potentially important effects have not been explored to date. The small size of the nanotube devices also allows the integration of the devices into living organisms. This will allow in-vivo electronic detection of biological processes.

Having thus described a preferred embodiment of a nanotube sensor for selective sensing of biomolecules, and a method for constructing it, it should be apparent to those skilled in the art that certain advantages of the within system have been achieved. It should also be appreciated that various modifications, adaptations, and alternative embodiments thereof may be made within the scope and spirit of the present invention. For example, a cellular membrane device has been illustrated, but it should be apparent that the inventive concepts described above would be equally applicable to devices that make use of other combinations with cellular components. For example, it should be apparent that the nanobioelectronic devices may include components which simulate the properties and functions of cellular components, such as artificial membranes, receptors, transport pores and the like, without departing from the spirit of the invention. The invention is defined by the following claims.

What is claimed is:

1. A nanoelectronic device, comprising:
a substrate;
at least one nanostructure disposed adjacent the substrate;
at least a first electrode arranged adjacent the substrate and in electrical communication with the nanostructure; and
at least a portion of a biological cell engaged with the nanoelectronic device, so as render the nanoelectronic device electrically responsive to a biomolecule analyte.

2. The nanoelectronic device of claim 1, wherein the at least one nanostructure comprises at least one carbon nanotube.

3. The nanoelectronic device of claim 1, wherein the at least one nanostructure comprises a network of nanotubes.

4. The nanoelectronic device of claim 1, wherein the nanoelectronic device further comprises at least a second electrode arranged adjacent the substrate and in electrical communication with the nanostructured element.

5. The nanoelectronic device of claim 1, wherein the nanoelectronic device further comprises at least a gate electrode disposed to influence an electrical property of the nanoelectronic device.

6. The nanoelectronic device of claim 1, wherein the nanoelectronic device further comprises at least a gate electrode disposed to influence an electrical property of the nanoelectronic device, and wherein the nanoelectronic device is electrically responsive to an electrical property of the at least one nanostructure under the influence of a gate voltage, the electrical property selected from the group consisting of transconductance and resistance.

7. The nanoelectronic device of claim 1, wherein the nanoelectronic device is electrically responsive to a capacitance of the least one nanostructured element.

8. The nanoelectronic device of claim 1, wherein the nanoelectronic device is configured to engage the at least a portion of a biological cell in an aqueous medium, and wherein the nanoelectronic device further comprises a reference electrode in electrical communication with the aqueous medium.

9. The nanoelectronic device of claim 1, wherein the at least a portion of a biological cell comprises an intact cell.

10. The nanoelectronic device of claim 1, wherein the at least a portion of a biological cell comprises a cell membrane.

11. The nanoelectronic device of claim 1, wherein the nanoelectronic device is configured to engage the at least a portion of a biological cell by direct contact of the nanostructure with the at least a portion of a biological cell.

12. The nanoelectronic device of claim 1, wherein the nanoelectronic device is configured to engage the at least a portion of a biological cell by the interaction of at least one intermediate functional molecule associated with the nanoelectronic device and the at least a portion of a biological cell.

13. The nanoelectronic device of claim 1, wherein the nanoelectronic device is configured to engage the at least a portion of a biological cell by the interaction of at least one intermediate functional molecule associated with the nanoelectronic device and the at least a portion of a biological cell, and wherein the at least one intermediate functional molecule comprises a binding species specific to a cell membrane structure.

14. The nanoelectronic device of claim 1, wherein the nanoelectronic device is configured to engage the at least a portion of a biological cell by interaction of the at least one nanostructure with a cytoplasmic side of a cell membrane.

15. The nanoelectronic device of claim 1, wherein the nanoelectronic device is configured to engage the at least a portion of a biological cell by the interaction of the at least one nanostructure with a extracellular side of a cell membrane.

16. The nanoelectronic device of claim 1, wherein the nanoelectronic device is configured to engage the at least a portion of a biological cell by the interaction of at least one intermediate functional molecule associated with the nanoelectronic device and the at least a portion of a biological cell, and wherein the at least one intermediate functional molecule includes a species enhancing a dipole effect of the engaging of the at least a portion of a biological cell.

17. The nanoelectronic device of claim 1, wherein the at least a portion of a biological cell includes a cell membrane of Halobacterium salinarum.

18. The nanoelectronic device of claim 1, further comprising an electrical measurement circuit in communication with the nanoelectronic device, the electrical measurement circuit measuring an electrical response of the nanoelectronic device.

19. The nanoelectronic device of claim 18, wherein the nanoelectronic device further comprises at least a gate electrode disposed to influence an electrical property of the nanoelectronic device, the electrical property selected from the group consisting of transconductance and resistance.

20. The nanoelectronic device of claim 19, wherein the electrical response comprises a change in the electrical property relative to a varying gate voltage applied by the gate electrode, the response selected from the group consisting of: a change in a threshold of the electrical property, and a change in a hysteresis of the electrical property.
